(19) United States
(12) Patent Application Publication
(10) Pub. No.: US 2024/0016550 A1
(43) Pub. Date: Jan. 18, 2024

(54) SCANNER FOR INTRAOPERATIVE APPLICATION

(71) Applicant: KICO KNEE INNOVATION COMPANY PTY LIMITED, New South Wales (AU)

(72) Inventors: Willy Theodore, Pymble (AU); Brad Peter Miles, Pymble (AU)

(73) Assignee: KICO KNEE INNOVATION COMPANY PTY LIMITED, New South Wales (AU)

(21) Appl. No.: 18/251,429

(22) PCT Filed: Nov. 1, 2021
(86) PCT No.: PCT/AU2021/051285

§ 371 Date: May 2, 2023

(30) Foreign Application Priority Data
Nov. 3, 2020 (AU) 2020904001

(51) Int. Cl. A61B 34/20; A61B 34/10; G06T 7/00; G06T 7/70; A61B 90/00

(52) U.S. Cl. CPC A61B 34/20; A61B 34/10; G06T 7/0012; G06T 7/70; A61B 90/39; A61B 2034/105; A61B 2034/107; A61B 2034/2057; A61B 2034/2068; A61B 2090/363; A61B 2090/3983; G06T 2200/24; G06T 2207/10028

(57) ABSTRACT

A tissue scanning system (1) comprising: a depth sensor (13) configured to determine distance to a surface; a pointer device (11), wherein the depth sensor (13) is mounted to the pointer device (11); a camera-based tracking system (3) configured to determine (114) relative orientation and position between an anatomical feature (9) and the pointer device (11); and at least one processing device (6). The processing device (6) is configured to: generate (116) a surface point cloud (16) of a surface (17) associated with the anatomical feature (9) based on a plurality of determined distances from the depth sensor (13) and corresponding relative orientation and position of the pointer device (11) relative to the anatomical feature (9).

### TECHNICAL FIELD

The present disclosure relates to a system and method of scanning tissue during surgery.

### BACKGROUND

It is important to accurately position surgical tools during surgery for effective treatment. Examples of surgery requiring accurate placements of surgical tools include surgery in relation to bones and joints, such as knee and hip replacement surgery. This may involve cutting, or otherwise shaping, bone and cartilage of the patient and securing implantable components thereto.

This requires the surgical tools to be accurately configured relative to the patient such that the surgical tool can operate in accordance with the surgical plan. This may involve apparatus and systems that assist the surgeon to guide the surgical tool to the desired position.

Consideration for anatomical features of a patient is important for the surgical plan and the resultant operation. Some of the anatomical features of the patient may be determined preoperatively based on medical imaging data, such as CT (X-ray computed tomography) or MRI (magnetic resonance imaging) images. These medical images may be analysed by a computer to construct a 3D model, such as a segmented mesh, that represents the imaging data.

However, such 3D modelled features are not always perfect and accurate. During an operation, a surgeon may find deviations from the modelled anatomic features compared to the actual anatomical features. For example, once skin and muscle are moved, the exposed bone and other tissue deviates from the 3D model used for the surgical plan. In other examples, some anatomical features may not be able to be accurately modelled preoperatively due to difficulties imaging that particular anatomical feature.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In the present description the term “position” with reference to a position of an element may include a position of the element in two, or three, dimensional space, where context permits may also include the orientation of the element.

### SUMMARY

A tissue scanning system comprising: a depth sensor configured to determine distance to a surface; a pointer device, wherein the depth sensor is mounted to the pointer device; a camera-based tracking system configured to determine relative orientation and position between an anatomical feature and the pointer device; and at least one processing device. The processing device is configured to: generate a surface point cloud of a surface associated with the anatomical feature based on a plurality of determined distances from the depth sensor and corresponding relative orientation and position of the pointer device relative to the anatomical feature.

In some examples, the tissue scanning system further comprise: one or more pointer markers attached to the pointer device; and one or more tissue markers attached to the anatomical feature. To determine relative orientation and position between the anatomical feature and the pointer device, the camera-based tracking system, or the at least one processing device, is further configured to: identify the pointer markers and tissue markers in one or more fields of view of the camera-based tracking system; and based on locations of the pointer markers and tissue markers in the field of view, calculate the relative orientation and position between the anatomical feature and the pointer device.

In some examples, the pointer markers (19) and the tissue markers (21) are ArUco fiducial markers.

In some examples of the tissue scanning system, the pointer device includes a guide tip, wherein the relative position of the guide tip to the depth sensor is fixed, or selectively fixed, during use.

In some examples of the tissue scanning system, a relative distance between the guide tip and the depth sensor is selected to be within a desired operating range of the depth sensor.

In some examples of the tissue scanning system, wherein the guide tip is configured to contact an index point on the surface associated with the anatomical feature, wherein the guide tip aids in maintaining a scanning distance between the depth sensor and the surface to be within the desired operating range.

In some examples of the tissue scanning system, the contact between the index point and the guide tip forms a pivot point such that as the pointer device is moved relative to the anatomical feature around the pivot point. The depth sensor determines a corresponding depth to the surface for that relative orientation and position to generate the surface point cloud of the surface.

In some examples, the pivot point is an intermediate reference point used to determine relative orientation and position of the anatomical features and the pointer device.

In some examples, the depth sensor is selected from one or more of: a Lidar (light detection and ranging); and/or an optical rangefinder.

In some examples, the tissue scanning system further comprise: a second camera mounted to the pointer device, wherein the depth sensor is directed in a direction within a field of view of the second camera; and a graphical user interface to display at least part of an image from the second camera.

In some examples, the at least one processing device of the tissue scanning system is further configured to: receive a patient profile of the anatomical feature); determine a predicted outline of the anatomical feature based on the patient profile; generate a modified image comprising the image from the second camera superimposed with the predicted outline; wherein the graphical user interface displays the modified image to guide a user to direct the depth sensor mounted to the pointer device to surface(s) corresponding to the predicted outline of the anatomical feature.

In some examples of the tissue scanning system, the processing device is further configured to: compare the generated surface point cloud with the patient profile; and generate an updated patient profile based on a result of the comparison.

In some examples of the tissue scanning system, the depth sensor and the at least one processing device are part of a mobile communication device.

There is also provided a method of acquiring a surface point cloud of a surface associated with an anatomical feature, the method comprising: receiving a plurality of determined distances from a depth sensor, wherein each determined distance has accompanying spatial data indicative of relative orientation and position of the depth sensor to the anatomical feature; determining the relative orientation and position of the depth sensor to the anatomical feature from the spatial data; and generating a surface point cloud of the surface associated with the anatomical features based on: the plurality of determined distances from the depth sensor: and corresponding relative orientation and position of the depth sensor to the anatomical feature.

In some examples of the method, determining relative orientation and position of the depth sensor to the anatomical feature further comprises: determining the spatial data by identifying in one or more fields of view of a camera-based tracking system: pointer markers mounted relative to the depth sensor; and tissue markers mounted relative to the anatomical feature. Based on locations of the pointer markers and tissue markers in the field of view, the method further includes calculating the relative orientation and position between the anatomical feature and the depth sensor.

In some examples, the method further comprises: receiving an image from a second camera, wherein the depth sensor is directed in a direction within a field of view of the second camera; receiving, a patient profile of the anatomical feature; determining a predicted outline of the anatomical feature based on the patient profile; generating a modified image comprising the image from a second camera superimposed with the predicted outline; displaying, at a graphical user interface, the modified image to guide a user to direct the depth sensor to surface(s) corresponding to the predicted outline of the anatomical feature.

In some examples, the method further comprises: comparing the generated surface point cloud with the patient profile; and generating an updated patient profile based on a result of the comparison.

A non-transitory, tangible, computer-readable medium comprising program instructions that, when executed, cause a processing device to perform the method.

There is also provided a tissue scanning system comprising: a camera-based tracking system configured to: determine relative orientation and position between an anatomical feature and a pointer device; and a depth sensor at the pointer device configured to capture surface point cloud measurements of surface(s) associated with the anatomical feature.

There is also provided a tissue scanning system comprising: a pointer device to receive a depth sensor configured to capture surface point cloud measurements of surface(s) associated with an anatomical feature; and a camera-based tracking system configured to: determine relative orientation and position between an anatomical feature and the pointer device.

### BRIEF DESCRIPTION OF DRAWINGS

FIG. 1 is a schematic of a tissue scanning system to acquire a surface point cloud associated with an anatomical feature;

FIG. 2 is a perspective view of a pointer device and depth sensor of the tissue scanning system in FIG. 1;

FIG. 3a is an image from a camera mounted to the pointer device, showing a portion of the anatomical feature and a guide tip of the pointer device;

FIG. 3b is a modified image having a superimposed predicted outline over the image of FIG. 3a;

FIG. 4 is a flow diagram of a method of acquiring a surface point cloud associated with the anatomical feature;

FIG. 5 is a flow diagram of steps to determine relative orientation and position between an anatomical feature and the pointer device from spatial data;

FIGS. 6a and 6b illustrate an example of the pointer device and depth sensor and the respective coordinate systems;

FIG. 7 illustrates an example image from a camera-based tracking system of the tissue scanning system;

FIG. 8 illustrates another example of an image from a camera-based tracking system that includes the tissue marker and pointer marker for determining relative orientation and position of the anatomical feature and pointer device;

FIG. 9 illustrates a representation of the surface point cloud of the surface generated by the tissue scanning system;

FIG. 10 is a diagram illustrating the various sequence of transforms to bring a scanned mesh of the anatomical feature to a frame of reference of the anatomical feature; and

FIG. 11 illustrates a schematic of a processing device.

### DESCRIPTION OF EMBODIMENTS

Overview

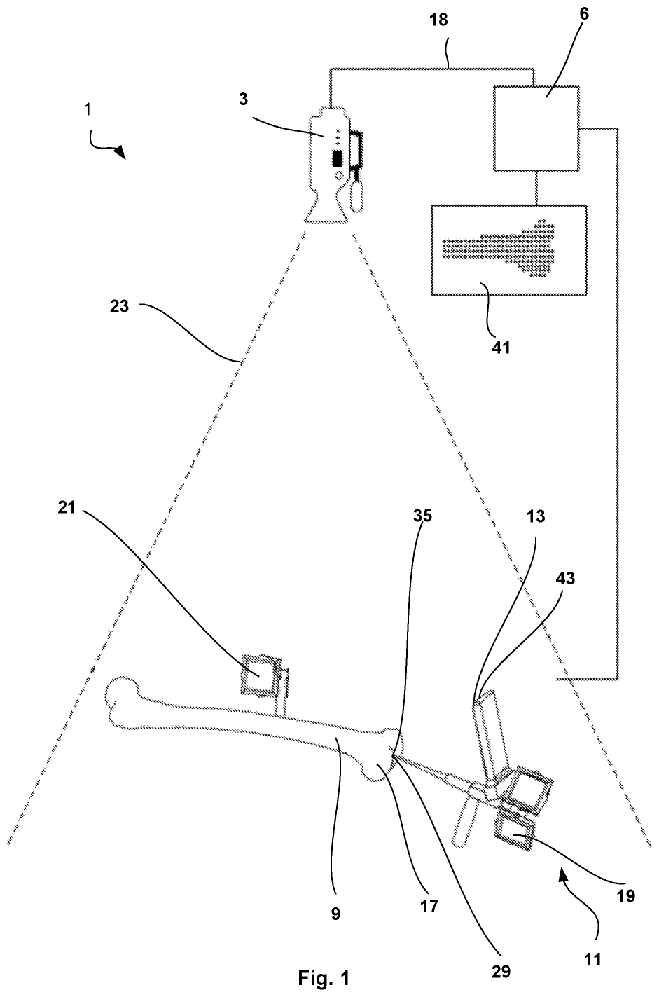

FIG. 1 illustrates an example of a tissue scanning system 1. This includes a depth sensor 13 configured to determine distance to a surface, in particular a surface 17 of an anatomical feature 9 of a patient. The depth sensor 13 is mounted to a pointer device 11. The pointer device 11 may include a guide tip 29 to contact an index point 35 on the surface 17 to aid locating the depth sensor 13 to within a desired operating range 33.

A camera-based system 3 is configured to determine 114 relative orientation and position between the anatomical feature 9 and the pointer device 11. This enables a processing device to generate 116 a surface point cloud 16 of the surface 17 based on associating the received 112 plurality of determined distances from the depth sensor 13 with the corresponding relative orientation and positions.

In some examples, the pointer device 11 has one or more pointer markers 19 and one or more tissue markers 21 attached to the anatomical feature 9. These pointer markers 19 and tissue markers 21, in the field of view 23 of the camera-based tracking system 3, assist in calculating the relative orientation and position between the anatomical feature 9 and the pointer device 11.

The tissue scanning system 1 may be used in a method 100 to capture information to generate the surface point cloud 16, or other three-dimensional model, of the anatomical feature during an operation. In some other examples, the tissue scanning system 1 is used to supplement, or update, an existing model or medical images of the anatomical feature. For example, pre-operative medical images may have been used to generate a patient profile 51 for that patient's specific anatomical feature, which is incorporated into a surgical plan. The tissue scanning system 1 may be used to directly scan the tissue during operation to provide an updated and more accurate surface point cloud 16 for the patient profile.

In some examples, the scanning system 1 may include providing, on a graphical user interface, a modified image to guide a user to the depth sensor 13 to the surface. This modified image is based on a real-time, or near real-time, image 49 superimposed with a predicted outline of the anatomical feature 9 based on a patient profile 51 (where the patient profile 51 may include information from preoperative medical imaging). This modified image can assist the user to direct the depth sensor to particular areas of interest to update the patient profile. This can be useful where particular tissue(s) are difficult to accurately image preoperatively.

The components of the tissue scanning system 1 will now be described in detail followed by methods of implementation.

The Anatomical Features and Tissue Markers

The anatomical features 9 can include bone, cartilage and other tissue of a patient. The surface 17 of the anatomical feature 9 can be any surface of interest dependent on the type of surgery. In some examples, this can include the surface of the femur bone (and related tissue) during arthroplasty.

To assist determining relative orientation and position, one or more tissue markers 21 can be attached to the anatomical feature 9. In the example of FIG. 1, the tissue marker 21 is attached to the femur bone, and in particular at a shaft portion that will not be removed during arthroplasty.

Although only one tissue marker 21 is illustrated in FIG. 1, it is to be appreciated that multiple tissue markers 21 can be used that may improve accuracy and range for the camera-based tracking system 3. Details of the tissue marker 21 will be described in further detail below and can include features similar to the pointer markers 10.

In some other examples, tissue markers 21 may not be necessary if the camera-based tracking system 3 can determine the orientation and position of the anatomical feature 9. The camera-based tracking system 3 may determine, from image(s), unique surfaces, outline, or other characteristics of the anatomical feature to determine the orientation and position.

Pointer Device

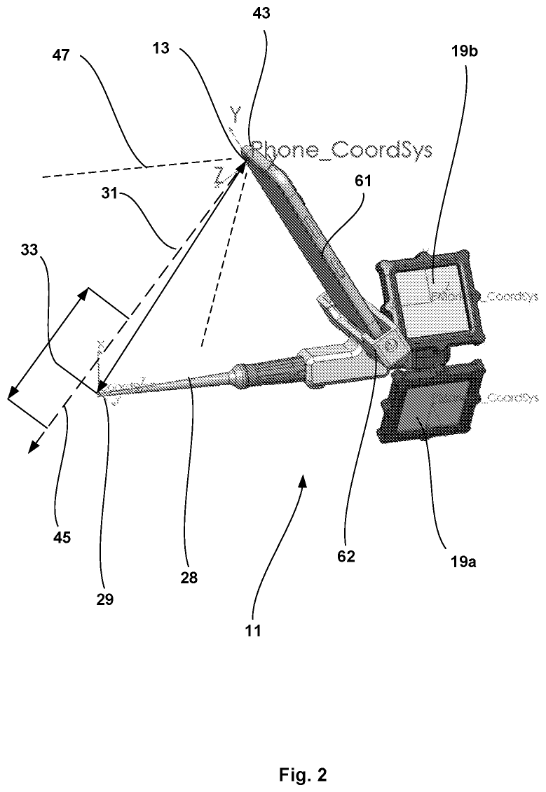

Referring to FIG. 2, the pointer device 11 is configured to receive the depth sensor 13. In this example, the depth sensor 11 is part of a mobile communication device 61 and the pointer device 11 is configured to receive the mobile communication device 61. This can include a cradle 62 to receive the mobile communication device 61. It is to be appreciated that the depth sensor 11 (or the mobile communication device 61) can be mounted by other means such as clamps, screws, and other fastening means.

The pointer device 11 also includes an elongated shaft 28 that terminates with a guide tip 29. The relative position of the guide tip 29 and the depth sensor 13 is fixed (or in alternative examples selectively fixed) during use. In some examples, the relative distance 31 between the guide tip 29 and the depth sensor 13 is selected to be within a desired operating range 33 of the depth sensor 13. The guide tip 29 can be used to contact an index point 35 on the surface 17 of the anatomical feature. Thus the guide tip 29 can aid in maintaining a scanning distance 37 between the depth sensor 13 and the surface 17 to be scanned to be within the desired operating range. 33.

Furthermore the contact between the index point 35 and the guide tip 29 can act as a pivot point 39. The user can apply slight pressure so that the guide tip 29 stays in contact with a particular index point 35 whilst the pointer device 11 is moved into various orientation and positions relative to the anatomical feature 9 around that pivot point 39. The depth sensor 13 determines the various depths that can be associated with those various relative orientation and positions for the system to generate the surface point cloud 16. In some examples, the guide tip 29 includes a sharp point to mildly pierce and engage the surface 17 so that the guide tip 29 does not slip from the particular index point 35. In other alternatives, the guide tip 29 may include a partially spherical surface to assist in rotation of the pointer device 11 around the pivot point 39.

The pointer device 11 may have one or more pointer markers 19 attached. The pointer markers 19 may assist the camera-based tracking system 3, and/or one or more processing devices 6 in the system to determine the orientation and position of the pointer device 11. This will be discussed in further detail below.

Depth Sensor

The depth sensor 13 is configured to determine a distance to a surface. This can include a depth sensor using laser light, such as a Lidar (light detection and ranging) technology or other laser range finder. This can involve determining distance by time-of-flight of a light pulse that is directed to the surface 17 and reflected back to the depth sensor 13.

In some examples, the depth sensor 13 can include a Lidar detector that can includes a flash Lidar to allow three dimensional image of an area with one scan. This can provide imaging Lidar technology that can determine a distance between the depth sensor 13 to a plurality of points on the surface 17. In some examples, the depth sensor (or processor processing distance data) has a range gate to ensure only certain measurements are associated with the measured surface point cloud. For example, it would be desirable to exclude the operating table, operating room floor, and walls, as these items do not relate to the anatomical features. Thus one parameter may include excluding measurements greater than or equal to a specified distance.

In some examples, the depth sensor 13 is associated with a mobile communication device, smart phone, tablet computer, or other electronic device. In one example, the depth sensor 13 is a Lidar scanner such as provided in the iPhone 12. Pro and the iPad Pro products from Apple Inc.

In some examples, the depth sensor 13 includes a depth camera. This can include a system including light projectors (including projectors that project multiple dot points) and a camera to detect reflections of those dot points on the surface 17, and a processor and software to create a surface point cloud 16, or other representation or data of a three dimensional surface. In some examples the depth sensor 13 includes the TrueDepth camera and sensor system in the iPhone (iPhone X, iPhone XS, iPhone 11 Pro, iPhone 12) offered by Apple Inc. In some examples, the system may utilise software to process data from the depth sensor, such as the Scandy Pro 3D Scanner offered by Scandy. Such software may, at least in part, also function to generate a 3D mesh, surface point cloud, or other 3D model. This can include a 3D model in STL file format.

In other examples, the depth sensor can include optical rangefinders that utilise trigonometry and a plurality of spaced-apart optical sensors to determine range. This can include utilising principles from coincidence rangefinders or stereoscopic rangefinders. Such a depth sensor can include two or more optical cameras with a known spaced-apart distance whereby features of a target object in the captured image(s) are compared. The deviations of the location of the features in the captured image(s) along with the known spaced-apart distance can be used to compute the distance between the depth sensor 13 and the surface 17.

Camera-Based Tracking System

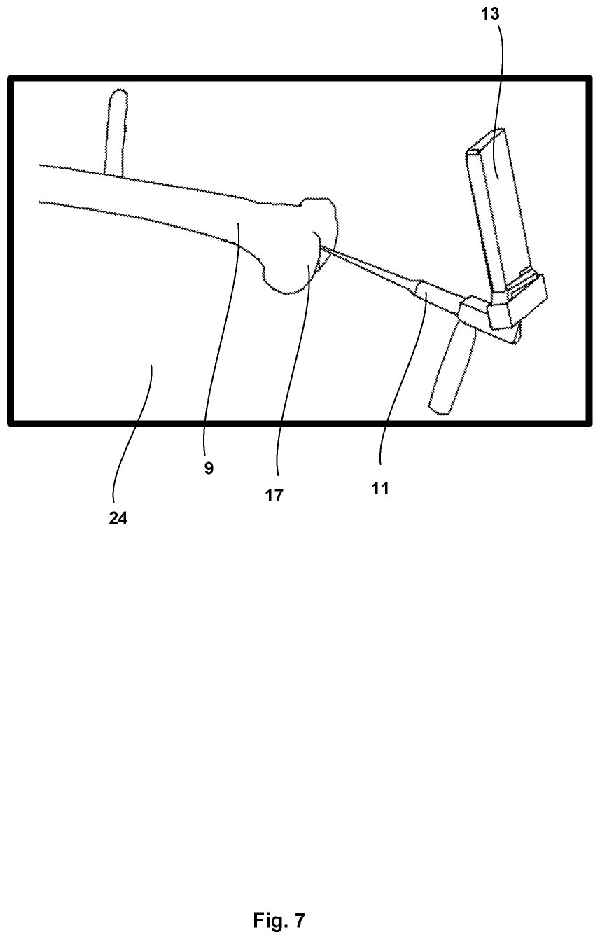

The camera-based tracking system 3 is configured to determine the relative orientation and position between the anatomical feature 9 and the pointer device 11. In one example of the camera-based tracking system 3, as illustrated in FIG. 1, this includes a camera with a field of view 23 that, in use, can detect at least part of the anatomical feature 9 and the pointer device 11 (or the corresponding tissue markers 21 and pointer marker 19). FIG. 7 illustrates an image 24 from the field of view 23 of the camera-based tracking system 3

In some examples, the camera-based tracking system 3 can include multiple cameras to provide a plurality of fields of view 23. This can assist in providing greater accuracy or enable the system to be more robust. This can include enabling the camera-base tracking system 3 to continue operating even if the anatomical feature, pointer device 11, or markers 19, 21 are masked from the field of view from one of the cameras. Such masking may occur, for example, from the body of the surgeon or other instruments in the operating theatre. In these examples, the location of the camera(s) of the camera-based tracking system 3 is known and may be used to define, at least in part, a frame of reference for the system to enable determination of relative orientation and position of the anatomical feature 9 and the pointer device 11.

In some examples, the camera-based tracking system 3 identifies markers 19, 21 in the field of view 23, whereby the markers can be more identifiable and distinguishable. For example the markers can include shape, colour, patterns, codes, or other unique or distinguishing features. In particular features that are in contrast to what would be found in the background of an operating room. In some example, this can include fiducial markers that can be used for identifying markers 19, 21 and for calculating a position or point in space of the marker 19, 21 in the field of view 23. In some examples, the fiducial marker may have features to enable determining the orientation of the marker. For example, the perceived shape of the marker (from the perspective of a camera) may be skewed depending on the relative orientation to the camera and these characteristics used to calculate the relative orientation of the marker. Determining the position and/or orientation of markers enables calculation of corresponding positions and orientations for the anatomical feature or pointer device.

In some examples, multiple markers are used. Referring to FIG. 2, the pointer markers 19 include two markers 19a, 19b that are provided at different positions at the pointer device 11. The use of multiple markers 19a, 19b enable two corresponding locations of the two markers 19a, 19b to be determined. With known relative positions between the markers 19a, 19b at the pointer device 11, such information can be used to calculate relative orientation of the pointer device 11.

In some examples, the multiple markers 19a, 19b include marks that are presented at different angles. This can be useful in some situations where the pointer device is orientated so that the one of the markers 19a, 19b is obscured or masked from the camera. The other marker, being orientated differently, may still be presented to visible to the camera-based tracking system.

In yet other examples, the different orientation of the markers 19a, 19b can aid the camera-based tracking system to calculate the orientation of the pointer device 11 (or anatomical feature 9) associated with the markers.

In some examples, the fiducial makers are ArUco, ARTag, ARToolKit, and/or AprilTag, fiducial markers that have been used for augmented reality technologies.

The camera-based tracking system 3 includes a processing device 6 to identify, in images captured in the field of view 23, the markers 19, 21 and their respective locations 25, 27. The processing device 6 can, based on those locations, 25, 27, calculate the relative orientation and positions between the anatomical features 9 and the pointer device. This calculation of relative orientation and position can resolving the multiple frames of reference and relative position and orientation of components in the system. For example:
(1) Relative position and orientation between the anatomical feature 9 and the tissue marker 21;
(2) Relative position and orientation between the tissue marker 21 and the camera-based tracking system 3;
(3) Relative position and orientation between the camera-based tracking system 3 and the pointer marker 21;
(4) Relative position and orientation between the pointer marker 19 and the pointer device 11;
(5) Relative position and orientation between the pointer device 11 and the depth sensor 13.
(6) Relative position between the depth sensor 13 and the surface 17 based on the distance measured by the depth sensor 13.

In some examples, the processor 6 performs a subset of the calculations noted above, and passes data to another processor to complete the calculations. In other examples, some calculations can be reduced. For example items (2) and (3) above may include a calculation of the relative position and orientation between the pointer marker 19 and the tissue marker 21 without using a frame of reference of the camera-based tracking system. This may be achieved when both the pointer markers 19 and the tissue marker 21 are both within the same field of view 23 of a camera.

In some examples, the camera-based tracking system may simply send images from the camera to another processing device to determine calculate the relative orientations and positions.

Second Camera and Graphical User Interface

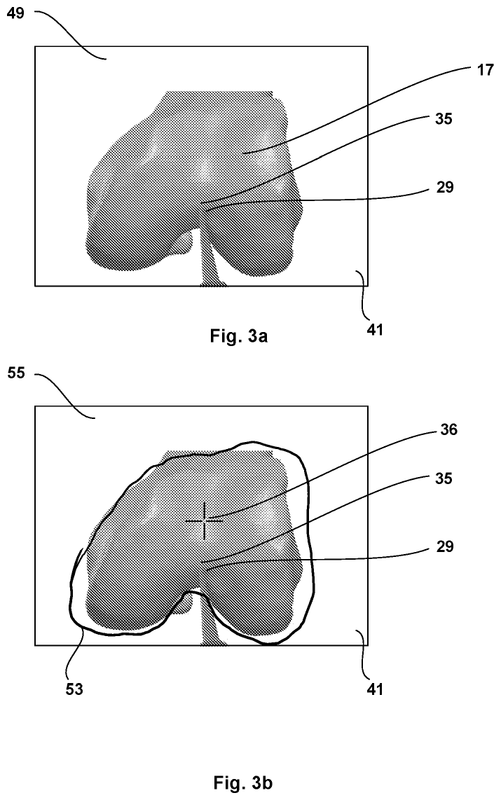

In some examples, a second camera 43 is mounted to the pointer device 11. The second camera 43 has a field of view 47 and the depth sensor 13 is directed in a direction 45 within that field of view 47. This allows the second camera 43 to capture an image 49 of the region that the depth sensor 13 is sensing which, in use, will include the surface 17 of the anatomical feature as illustrated in FIG. 3a.

A graphical user interface 41 can display at least part of the image 49 from the second camera 43 that can assist the surgeon in guiding the depth sensor 13 to various parts of the surface 17. In some examples, the graphical user interface 41 can include a reticle 36 to mark the portion that the depth sensor 13 is actively sensing.

The graphical user interface 41 may also display a virtual guide to the user for manipulating the pointer device 11/depth sensor 13 to enable measurements in specified areas of interest.

In some examples, the system includes a processing device 6 to generate a virtual guide for the user at the graphical user interface 41. This may include a receiving 105 a patient profile 51 of the anatomical feature 9. The patient profile 51 may comprise of earlier scans or models of the patient's anatomical feature. Such an initial patient profile 51 may be created pre-operatively from medical imaging, and/or with idealised models of such anatomical features. Such initial patient profiles 51 may not be precise, and thus the tissue scanning system 1 is used to update the patient profile 51 with refined measurements intra-operatively.

The processing device 6 then determines 107 a predicted outline 53 of the anatomical feature 9 based on the patient profile 51. The predicted outline 53 is from the perspective of the second camera 43 relative to the surface 17 of the anatomical feature 9. In some examples, data from the camera-based tracking system 3 can be used, at least in part, to determine the relative orientation of the anatomical feature 9 to the second camera 43. This relative orientation, in conjunction with the patient profile 51 can then be used to determine the perspective and the predicted outline 53.

The processing device 6 can then generate a modified image 55 (as illustrated in FIG. 3b) that includes: the image 49 from the second camera 43; and the predicted outline 53 superimposed on the image 49. This modified image 55, displayed at the graphical user interface 41 can be used by the surgeon to guide the depth sensor to the corresponding predicted outline 53. The advantage is to obtain more accurate, and actual, measurements of the surface 17 of the anatomical features during surgery with the determined surface point cloud 16.

The processing device 6 can compare 121 the generated surface point cloud 16 with the patient profile 51. The result of the comparison can be used to update the patient profile 51. An advantage of this system is to incorporate direct measurement of the surface 17 in the patient profile 51 that may be more accurate that a profile based only on medical imaging that may not have the same accuracy or resolution.

Mobile Communication Device

In some examples, the tissue scanning system 1 can include the use of a mobile communication device 61, such as a smart phone. For example, the iPhone 12. Pro offered by Apple Inc includes a processing device, communications interface, a Lidar sensor (that is a depth sensor), multiple cameras (that can be function as, or augment, the second camera 43 and/or camera-based tracking system 3). The mobile communication device 61 may also include inertial measurement sensors such as gyroscopes, accelerometers, and magnetometers that can assist in calculating orientation and movement of the depth sensor 13 and/or pointer device 11.

The mobile communication device 61 also includes a graphical user interface 41 that can display the image 49, the modified image 55, as well as other data including representations of the patient profile 51 and updated patient profile 59.

In some examples, one or more, or all of the functions of the processing device 6 are performed by the processing device in the mobile communication device 61. In other examples, data from the depth sensor, cameras and/or inertial measurement sensors are sent, via the communication interface, to another processing device to perform the steps of the method.

Method of Acquiring a Surface Point Cloud

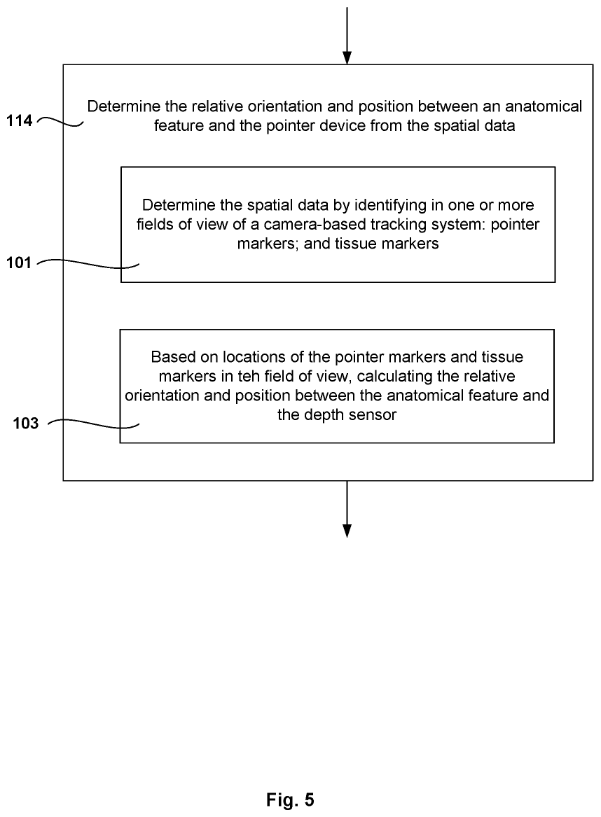

Referring to FIG. 5, an example of acquiring a surface point cloud 16 of a surface 17 of the anatomical feature will be described in detail. It is to be appreciated that this is a specific example and variations of the method 100 may have less steps or additional steps.

The method 100 includes receiving 112 a plurality of determined distances from the depth sensor 13 as the pointer device 11 is manipulated by the surgeon. This results in obtaining distance measurements at different locations on the surface 17. Each of the plurality of determined distances are associated with spatial data 18, where the spatial data is indicative of relative orientation and position of the depth sensor 13 to the anatomical feature 9. This is used to obtain each point that make up the surface point cloud 16

The spatial data 18 can be obtained from the camera-based tracking system 3. In one example, the spatial data 18 may include an image 24 (as shown in FIG. 7) that includes, within the field of view 23, the anatomical feature 9 and the pointer device 11. Based on this image, the relative orientation and position of the anatomical feature 9 and the pointer 11 can be calculated. Since the position of the depth sensor 13 at the pointer device is known, this can be used to determine 114 the relative orientation and position of the depth sensor 13 relative to the anatomical feature.

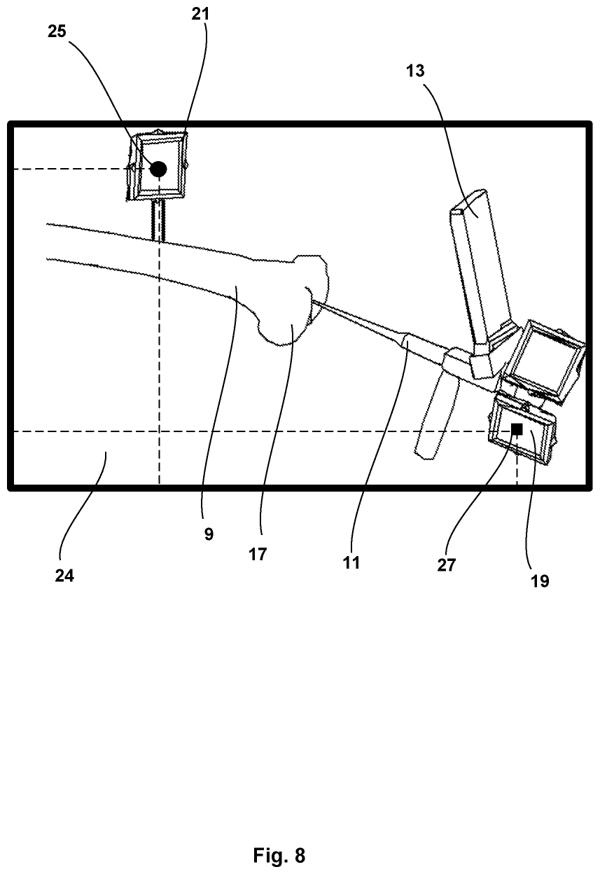

In another example, as illustrated in FIG. 5 and FIG. 8, the spatial data is based on identifying 101 pointer and tissue markers 19, 21 in the field of view 23 that are attached, respectively, to the pointer device 11 and anatomical feature. Based on the locations 25, 27, of the pointer markers 19 and tissue markers 21 in the field of view 23 (such as in image 24), the method includes calculating 103 the relative orientation and position between the anatomical feature 9 and the pointer device 11. This can also include calculating the relative position and orientation with the depth sensor 13).

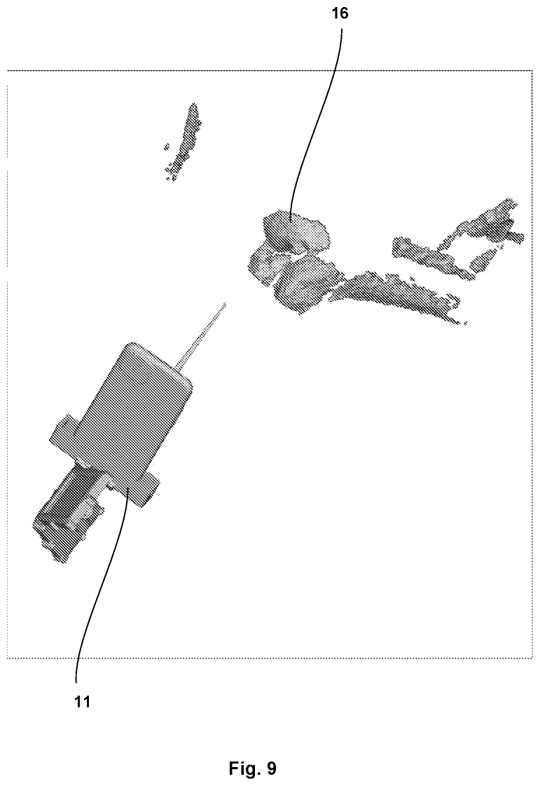

The method 100 further includes generating 116 a surface point cloud 16 of the surface 17 associated with the anatomical features 9 as represented in FIG. 9. The surface point cloud 16 is generated based on:
The plurality of determined distances from the depth sensor 13; and
The corresponding orientation and position of the depth sensor to the anatomical feature 9 for each of the determined distances.

FIG. 9 illustrates the surface point cloud 16 of parts of the surface 17 that have been scanned, whilst other portions that have not been scanned remain blank. A representation of the pointer device 11 is provided to show the spatial relationship of the pointer device 11 and this does not form part of the actual surface point cloud 16 of the surface 17.

In some examples, the step of generating 116 a surface point cloud 16 may include multiple steps. In one example, the depth sensor 13 and system may first determine a 3D mesh, 3D point cloud, or other 3D model of the surface 17 relative to the depth sensor 13. That is, using a frame of reference relative to the depth sensor 13 (which in some cases is the same as, or closely associated with, the frame of reference of the mobile communication device). The second step is to apply a transformation to a coordinate system desired by the system and user, which can include a coordinate system relative to a part of the pointer device, markers, anatomical feature 9 or even a reference point at the operating theatre. The transformation and selected coordinate system can allow easier relationships to be determined and calculated with respect to the anatomical features 9 (e.g. frame of reference relative to the femur bone).

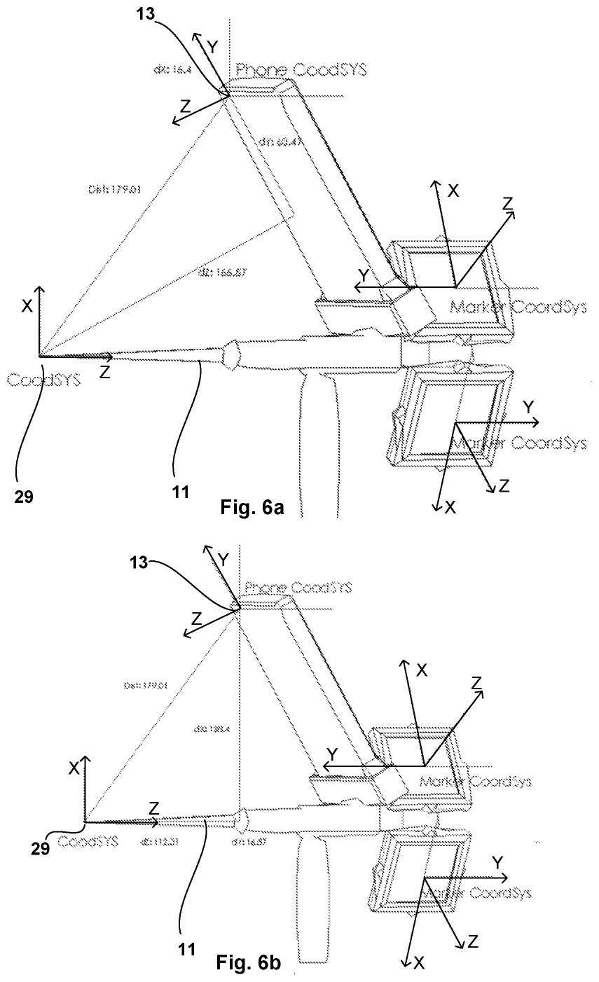

FIG. 6a illustrates various coordinate systems including: the depth sensor 13 coordinate system (that coincides with mobile communication device coordinate system), the pointer coordinate system, and marker 19 coordinate system. The depth sensor 13 may output distance data in a coordinate system relative to the depth sensor 13. However, the system may desire distance data relative to another coordinate system, say the coordinate system of the pointer device relative to the pointer tip 29. In one example, this includes applying a transformation matrix to the data for rotation and translations.

The normal vector of the coordinate system of the depth sensor relative to the pointer device is, in this example, defined by a plane: where x-coord: 16.4 mm, y-coord −63.5 mm, and z-coord 166.6 mm. This produces the transform matrix below:

| Name | | | | | |
|---|---|---|---|---|---|
| 2 | Group | Generation | Colour | Style | |
| Plane Origin | None | 0 | Fore ground | Constructi [symbol] | Le [symbol] |

| | | | |
|---|---|---|---|
| Origin: | X-coord | Y-coord | Z-coord |
| | 16.4357 | −63.4740 | 166.5607 |
| X Vector: | i | j | k |
| | 0.0018 | 0.8653 | −0.5013 |
| Y Vector: | i | j | k |
| | 1.0 | −0.0016 | 0.0008 |
| Z Vector: | i | j | k |
| | −0.0001 | −0.5013 | −0.8653 |

[symbol]

indicates data missing or illegible when filed

To account for the translation of the depth sensor 13 to the pointer coordinate system, transformation further includes a correct translation of: x: 138.4 mm, y: −16.57 mm, z: 112.31 mm as illustrated in FIG. 6b.

In some examples, the system may require a sequence of transformation to bring the various data in to a single desired coordinate system. In some particular examples, this includes transforming the data to be in the reference frame of the anatomical feature 9, such as the bone.

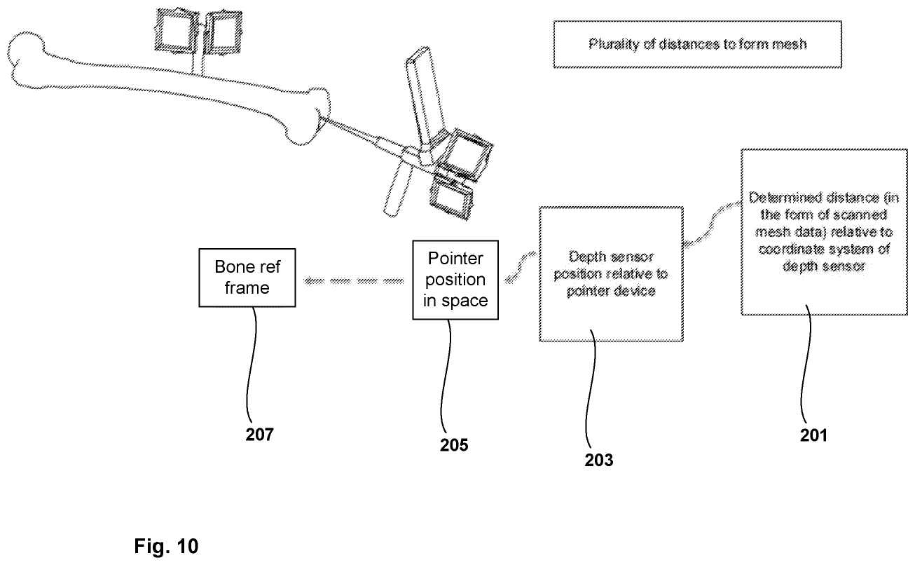

Referring to FIG. 10, this can include the depth sensor 13 and system determining 201 a plurality of distance measurements (which may be in raw form, or processed into, at least in part a 3D mesh or point cloud) that are relative to a coordinate system of the depth sensor 13 (or in the case of a mobile communication device the coordinate system specified by that device). That information needs to be transformed 203 to a coordinate system relative to the pointer device 11 (an example of which is discussed above). That information, in turn needs to be transformed 207 to a common frame of reference, namely that of the bone. In some examples this includes the camera-base tracking system 3 determining 205 locations in space of the respective pointer markers 19 and tissue markers 21 so that appropriate transformations can be applied to the data so that the surface point cloud 16 can be generated relative to a frame of reference of the anatomical feature 9.

In some examples, the system may, at least in part, use the pivot point 39 as an intermediate reference point to assist in calculating an appropriate translation for the transform. For example, the pivot point 39 (calibrated to a known position and orientation to the depth sensor 13) may, in use, be in contact with the surface of the anatomical feature.

Method of Visually Guiding the Pointer Device

The method 100 may also include providing a visual guide at a graphical user interface 41. This can aid the user to scan particular areas of interest and/or areas that have not been adequately scanned.

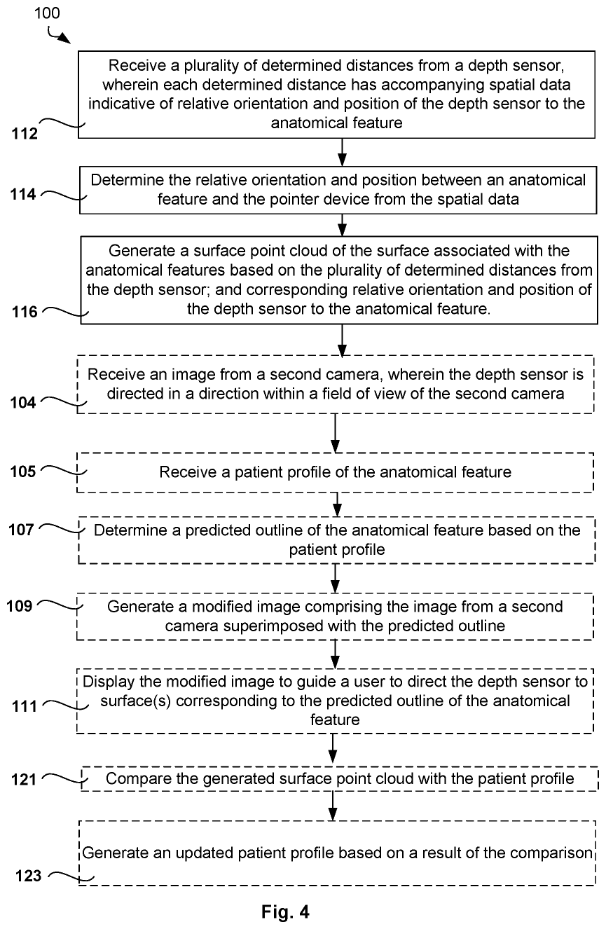

Referring back to FIG. 4, the method further includes receiving an image 49 from the second camera 43, wherein the depth sensor 13 is directed in a direction 45 within a field of view 47 of the second camera 43. Referring to FIG. 3a that illustrates an example of the image 49 from the second camera 43, this shows the surface 17 of the anatomical feature 9 and the guide tip 29 of the pointer device 11.

The method further includes receiving 105 a patient profile 51 of the anatomical feature 9. This patient profile may be constructed with medical imaging data of the patient, models of the patient based on the medical images and/or other scans and measurements of the patient, idealised or approximate models of anatomical features of a human. The method further includes determining 107 a predicted outline 53 of the anatomical feature based on the patient profile, which takes into consideration the perspective that the second camera 43 is viewing the anatomical feature 9.

As illustrated in FIG. 3B, a modified image 55 is generated 109 and displayed 111, whereby the modified image 55 comprises at least part of the original image 49 of the anatomical features superimposed with the predicted outline 53. The modified image 55 may also include a reticle 36 that represents the portion/direction 45 in the field of view 47 that the depth sensor 13 will be scanning.

In this example, it is desirable for the surgeon to obtain a more accurate data on the outer surface 17 of the anatomical feature 9. As such, the user can then manipulate the pointer device 11 so that the reticle 36 is at or around the predicted outline 53 and the user can then trace (i.e. follow) the predicted outline 53 to obtain measurements along that predicted outline, which in turn causes a surface point cloud 16 to be generated for that corresponding traced area of the surface 17.

In this example, the predicted outline 53 is in the form of a substantially enclosed loop. However, it is to be appreciated that in other examples, the predicted outline could be a silhouette of an area, whereby the silhouette guides the user to scan an area of interest.

In some examples, the surface point cloud 16 is used to update a patient profile 59. In one example, the surface point cloud 16 becomes at least part of the updated patient profile 59.

In other examples, the method includes comparing 121 the generated surface point cloud 16 with the existing patient profile. That is, identifying the points of difference between the patient profile 51 on record and scanned surface point cloud 16. Then, the method includes updating the updated patient profile 59 based on a result of the comparison. By using comparisons, this may be useful in cases where only a portion of the patient profile is scanned and updated.

Other Features

The mobile communication device 61 and the processor therein may perform the majority, or all, of the steps of the method 100 described above. However, it is to be appreciated that the mobile communication device 61 can send outputs to other devices via one or more communications networks (including wireless networks) to another device. For example, the user may wish to use an alternative graphical user interface 41 (such as a larger display screen in the operator). To that end, the contents displayed at the screen of the mobile communication device 61 may be mirrored to that other display. In other examples, the output may include sending (including) streaming, images 49 and modified images 55 to other devices. In yet other examples, data associated with the patient profile, or updated patient profile can be sent and stored at a storage device in communication with the mobile communication device 61. This includes storing the data on cloud based storage.

Variations

In some examples the camera-based tracking system may utilise one or more cameras (which may include the second camera 43) at a mobile communication device. For example, a forward facing camera of the mobile communication device is configured to locate, in the field of view, the tissue marker 21 at the anatomical feature 9. The same camera, or another camera, may be used to identify pointer markers 19 at the pointer device. This information can be used to determine information of the relative orientation and position of the pointer device 11 and, ultimately, the depth sensor 13.

In some examples, the tissue scanning system may include a kit comprising: the pointer device 11 and the camera-based tracking system 3. The pointer device 11 is configured to receive a depth sensor (13) configured to capture surface point cloud measurements of surface(s) associated with an anatomical feature. For example, the pointer device 11 is configured to receive a separately supplied mobile communication device having a depth sensor.

In other examples, the tissue scanning system may include a kit comprising: the camera-based tracking system 3 and the depth sensor 13. The depth sensor 13 is located at a pointer device. The pointer device 11 configured to receive the mobile communication device and to aid directing and locating the depth sensor 13 may be supplied separately.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

Processing Device

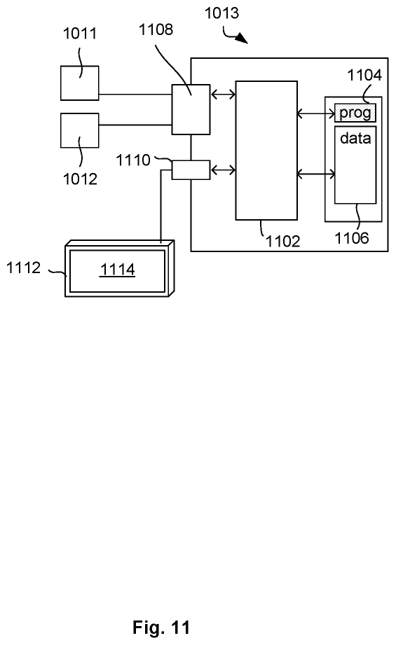

The processing device 1013, as illustrated in FIG. 11, includes a processor 1102 connected to a program memory 1104, a data memory 1106, a communication port 1108 and a user port 1110. The program memory 1104 is a non-transitory computer readable medium, such as a hard drive, a solid state disk or CD-ROM. Software, that is, an executable program stored on program memory 1104 causes the processor 1102 to perform the method 100 in FIG. 4.

The processor 1102 may receive determined distances and orientation and position data and store them in data store 1106, such as on RAM or a processor register. The depth sensor data and other information may be received by the processor 1102 from data memory 106, the communications port 1108, the input port 1011, and/or the user port 1110.

The processor 1102 is connected, via the user port 1110, to a display 1112 to show visual representations 1114 the images, or modified images from cameras, and/or the surface point cloud. The processor 1102 may also send the surface point cloud, as output signals via communication port 1108 to an output port 1012.

Although communications port 1108 and user port 1110 are shown as distinct entities, it is to be understood that any kind of data port may be used to receive data, such as a network connection, a memory interface, a pin of the chip package of processor 1102, or logical ports, such as IP sockets or parameters of functions stored on program memory 1104 and executed by processor 1102. These parameters may be stored on data memory 1106 and may be handled by-value or by-reference, that is, as a pointer, in the source code.

The processor 1102 may receive data through all these interfaces, which includes memory access of volatile memory, such as cache or RAM, or non-volatile memory, such as an optical disk drive, hard disk drive, storage server or cloud storage. The processing device 13 may further be implemented within a cloud computing environment, such as a managed group of interconnected servers hosting a dynamic number of virtual machines.

1. A tissue scanning system (1) comprising:
a depth sensor (13) configured to determine distance to a surface;
a pointer device (11), wherein the depth sensor (13) is mounted to the pointer device (11);
a camera-based tracking system (3) configured to determine (114) relative orientation and position between an anatomical feature (9) and the pointer device (11); and
at least one processing device (6) configured to:
generate (116) a surface point cloud (16) of a surface (17) associated with the anatomical feature (9) based on a plurality of determined distances from the depth sensor (13) and corresponding relative orientation and position of the pointer device (11) relative to the anatomical feature (9).

2. A tissue scanning system (1) according to claim 1, further comprising:
one or more pointer markers (19) attached to the pointer device (11); and
one or more tissue markers (21) attached to the anatomical feature (9);
wherein to determine (114) relative orientation and position between the anatomical feature (9) and the pointer device (11), the camera-based tracking system (3), or the at least one processing device (6), is further configured to:
identify (101) the pointer markers (19) and tissue markers (21) in one or more fields of view (23) of the camera-based tracking system (3); and
based on locations (25, 27) of the pointer markers (19) and tissue markers (21) in the field of view (23), calculate (103) the relative orientation and position between the anatomical feature (9) and the pointer device (11).

3. A tissue scanning system (1) according to any one of the preceding claim wherein the pointer markers (19) and the tissue markers (21) are ArUco fiducial markers.

4. A tissue scanning system (1) according to any one of the preceding claims wherein the pointer device (11) includes a guide tip (29), wherein the relative position of the guide tip (29) to the depth sensor (13) is fixed, or selectively fixed, during use.

5. A tissue scanning system (1) according to claim 4, wherein a relative distance (31) between the guide tip (29) and the depth sensor (13) is selected to be within a desired operating range (33) of the depth sensor (13).

6. A tissue scanning system (1) according to claim 5, wherein the guide tip (29) is configured to contact an index point (35) on the surface (17) associated with the anatomical feature (9), wherein the guide tip (29) aids in maintaining a scanning distance (37) between the depth sensor (13) and the surface (17) to be within the desired operating range (33).

7. A tissue scanning system according to claim 6, wherein the contact between the index point (35) and the guide tip (29) forms a pivot point (39) such that as the pointer device (11) is moved relative to the anatomical feature (9) around the pivot point (39), and the depth sensor determines a corresponding depth to the surface (17) for that relative orientation (5) and position (7) to generate the surface point cloud (16) of the surface (17).

8. A tissue scanning system according to claim 7, wherein the pivot point (39) is an intermediate reference point used to determine relative orientation (5) and position (7) of the anatomical features (9) and the pointer device (11).

9. A tissue scanning system according to any one of the preceding claims wherein the depth sensor is selected from one or more of:
a Lidar (light detection and ranging); and/or
an optical rangefinder.

10. A tissue scanning system (1) according to any one of the preceding claims further comprising:
a second camera (43) mounted to the pointer device (11), wherein the depth sensor (13) is directed in a direction (45) within a field of view (47) of the second camera (43); and
a graphical user interface (41) to display at least part of an image (49) from the second camera (43).

11. A tissue scanning system (1) according to claim 10, wherein the at least one processing device (6) is further configured to:
receive (105) a patient profile (51) of the anatomical feature (9);
determine (107) a predicted outline (53) of the anatomical feature (9) based on the patient profile (51); and
generate (109) a modified image (55) comprising the image (49) from the second camera (43) superimposed with the predicted outline (53);
wherein the graphical user interface (41) displays the modified image (55) to guide a user to direct the depth sensor (13) mounted to the pointer device (11) to surface(s) (17) corresponding to the predicted outline (53) of the anatomical feature (9).

12. A tissue scanning system (1) wherein the processing device (6) is further configured to:
compare (121) the generated surface point cloud (16) with the patient profile (51); and
generate (123) an updated patient profile (59) based on a result of the comparison.

13. A tissue scanning system (1) according to any one of claims 1 to 12, wherein the depth sensor (13) and the at least one processing device (6) are part of a mobile communication device (61).

14. A method (100) of acquiring a surface point cloud (16) of a surface (17) associated with an anatomical feature (9), the method comprising:
receiving (112) a plurality of determined distances from a depth sensor (13), wherein each determined distance has accompanying spatial data (18) indicative of relative orientation and position of the depth sensor (13) to the anatomical feature (9);
determining (114) the relative orientation and position of the depth sensor (13) to the anatomical feature (9) from the spatial data (18); and
generating (116) a surface point cloud (16) of the surface (17) associated with the anatomical features (9) based on:
the plurality of determined distances from the depth sensor (13); and
corresponding relative orientation and position of the depth sensor to the anatomical feature (9).

15. A method (100) according to claim 14, wherein determining (114) relative orientation and position of the depth sensor (13) to the anatomical feature (9) further comprises:
determining the spatial data (18) by identifying (101) in one or more fields of view (23) of a camera-based tracking system:
pointer markers (19) mounted relative to the depth sensor (13); and
tissue markers (21) mounted relative to the anatomical feature (9),
based on locations (25, 27) of the pointer markers (19) and tissue markers (21) in the field of view (23), calculating (103) the relative orientation and position between the anatomical feature (9) and the depth sensor (13).

16. A method according to either claim 14 or 15, further comprising:
receiving (104) an image (49) from a second camera (43), wherein the depth sensor (13) is directed in a direction (45) within a field of view (47) of the second camera (43);
receiving (105), a patient profile (51) of the anatomical feature (9);
determining (107) a predicted outline (53) of the anatomical feature (9) based on the patient profile (51);
generating (109) a modified image (55) comprising the image (49) from a second camera (43) superimposed with the predicted outline (53); and
displaying (111), at a graphical user interface (41), the modified image (55) to guide a user to direct the depth sensor (13) to surface(s) (17) corresponding to the predicted outline (53) of the anatomical feature (9).

17. A method according to claim 16 further comprising:
comparing (121) the generated surface point cloud (16) with the patient profile (51); and
generating (123) an updated patient profile (59) based on a result of the comparison.

18. A tissue scanning system (1) comprising:
a camera-based tracking system (3) configured to:
determine relative orientation (5) and position (7) between an anatomical feature (9) and a pointer device (11); and
a depth sensor (13) at the pointer device (11) configured to capture surface point cloud measurements (15) of surface(s) (17) associated with the anatomical feature (9).

19. A tissue scanning system (1) comprising:
a pointer device (11) to receive a depth sensor (13) configured to capture surface point cloud measurements of surface(s) associated with an anatomical feature; and
a camera-based tracking system (3) configured to:
determine relative orientation and position between an anatomical feature and the pointer device.

20. A non-transitory, tangible, computer-readable medium comprising program instructions that, when executed, cause a processing device to perform the method of any one of claims 14 to 17.
